(19) United States
(12) Patent Application Publication
(10) Pub. No.: US 2003/0044853 A1
(43) Pub. Date: Mar. 6, 2003

(54) Method for conducting cell-based analyses using laminar flow, and device therefor

(76) Inventors: David Socks, Manhattan Beach, CA (US); Thomas Ahl, Cupertino, CA (US)

Correspondence Address: REED & ASSOCIATES, 800 MENLO AVENUE, SUITE 210, MENLO PARK, CA, 94025, US

(21) Appl. No.: 09/941,944

(22) Filed: Aug. 28, 2001

(51) Int. Cl. G01N 33/53; G01N 33/542; C12M 1/34

(52) U.S. Cl. 435/7.9; 435/287.2

(57) ABSTRACT

The invention pertains to devices and methods relating to the formation of one or more fluid lanes on a substrate surface, to expose a portion of a target region on the surface to one or more reagents. Typically, a flow passage is provided that is defined at least in part by a substrate having a target region on a surface thereof. One or more inlets each allows a fluid to be introduced in contiguous laminar flow through the flow passage to form a fluid lane downstream from the inlet over a portion of the target region. Typically, at least one inlet is position to allow a fluid to be introduced directly into the flow passage in a direction nonparallel to the flow passage. Typically, the inventive devices and methods may be employed to determine the effects of a plurality of candidate compounds on a monolayer of immobilized cells.

### TECHNICAL FIELD

The present invention relates to devices and methods for delivering one or more lanes of reagent to a substrate surface through laminar flow. More specifically, the invention relates to compact devices and methods that employ laminar flow fluid delivery for rapid and efficient cell-based analysis.

### BACKGROUND

In the field of drug discovery and combinatorial chemistry, a number of different types of technologies have been developed to carry out high-throughput screening to identify candidate compounds potentially exhibiting beneficial pharmacological properties. In molecular analysis, particularly nucleotidic biomolecular analysis, array technology has been extensively investigated. For example U.S. Pat. No. 5,700,637 to Southern et al. describes a method for generating an array of oligonucleotides of chosen lengths within discrete features on a support material. The method is described as involving segregating a support material into discrete feature locations and repeatedly coupling nucleotide precursors to sets of feature locations until the desired array has been generated. In addition, the formation of nucleotidic arrays of high density is also known in the art. See, e.g., U.S. Pat. No. 5,744,305 to Fodor et al. These arrays may be used to perform in rapid high-throughput screening of samples containing nucleotidic materials. Array technology may also be adapted to rapidly identify biomolecular compounds that are not nucleotidic as well.

Generally, high-throughput screening technology allows for rapid identification of a large number of compounds potentially having desired or beneficial pharmacological properties. However, such screening generally provides little information regarding the safety or efficacy of these candidate compounds in humans. Selecting which of these identified candidate compounds to pursue in clinical trials is currently the most time-consuming, labor intensive and inefficient stage of the drug discovery process.

Traditionally, animal studies are performed to evaluate the efficacy and toxicity of promising candidate compounds, since the effect of a candidate compound on animals often correlate well to the effect of the candidate compound in humans. Animal studies, however, are time-consuming and costly. In addition, excessive drug testing in animals is discouraged in many regions around the world including the United States and Europe. Thus, there is a need to carry out information-rich screening of candidate compounds before candidate compounds are tested on animals.

Cellular analysis data provide critical information to the understanding of the effect of candidate compounds on complex cell functions. Studies employing living cells, in particular, provide unique advantages in the evaluation of a candidate compound's pharmacological properties. Since living cells testing can often approximate animal testing, candidate drugs may be screened according to their interaction with cells. Conventional methods for analyzing drug-cell interactions, however, require a large number of cells and a large quantity of candidate compounds, either or both of which may not be readily available. In addition, cumbersome methods may be needed to effect precise control over fluid delivery associated with such cell analysis. Thus, there is a need for improved methods for cellular analysis that employ small quantities of cells and candidate compounds.

A number of patents disclose the use of cellular arrays for candidate compound screening. For example, U.S. Pat. Nos. 5,976,826 and 5,776,748 to Singhvi are related patents, each directed to a device for adhering at least one cell in a specific and predetermined pattern. The device includes a plate that defines a surface as well as a plurality of cytophilic islands, the surfaces on which cells may adhere. The cytophilic islands, formed from a self-adhesive monolayer, are isolated by contiguous cytophobic regions to which cells do not adhere. The cytophobic regions may be sufficiently wide to prevent cells adhered to the cytophilic islands from contacting each other, except via formation of cellular bridges above and free of adhesive contact with the cytophobic regions. In the alternative, the cytophobic regions may be sufficiently wide such that less than 10 percent of cells adhered to these cytophilic islands form bridges across said cytophobic regions and contact each other. U.S. Pat. No. 6,180,239 to Whitesides et al. describes that such an array may be formed by employing a stamp for imparting a pattern of the self-assembled monolayer of the molecular species on a surface.

More recently, U.S. Pat. No. 6,103,409 to Taylor describes a method for producing a cassette for cell screening. A base with a surface is provided, and a micropatterned chemical array is prepared. The micropatterned chemical array is modified to produce a modified micropatterned chemical array comprising multiple different cell binding locations on the surface of the base. The different cell binding locations interact with different cell types, and each cell-binding location comprises a well. Once cells are bound to the modified micropatterned chemical array to produce an ordered array of cell types seeded on the wells, a fluid delivery system is provided for delivering a combinatorial of reagents to the ordered array of cell types. The fluid delivery system is typical of many microfluidic devices in that it comprises a chamber that mates with the base containing the ordered array of cell types. The chamber comprises: (i) etched domains matching the wells on the surface of the base, and (ii) microfluidic channels that supply fluid to the etched domains.

Known miniaturized cellular assay technologies, such as that those described above, may be employed to evaluate the interaction between a candidate compound with a number of different types of cells, and/or the interaction of one type of cell with a number of different candidate compounds. In the former case, different types of cells are exposed to the same candidate compound. Thus, if array technology is employed to carry out cellular assay, the different types of cells must be controllably immobilized to appropriate array locations. In the latter case, different candidate compounds must be controllably delivered to different array locations. Thus, known cellular assay technology suffers from the drawback that it generally requires sophisticated cell placement equipment, complex fluid handling equipment, or both. As a result, known miniaturized cellular assay technology either exhibits a lowered throughput, high cost, or both.

For example, International Publication WO 00/56444 describes a method for producing an interaction between a hydrodynamically focused liquid (or a component of the hydrodynamically focused liquid) and a selected region of a target surface. Cells may be immobilized on the target surface. The method involves providing a target surface that defines, in part, a liquid flow path that uses two guidance streams to direct a flow of hydrodynamically focused liquid stream, which is then interposed between the liquid guidance streams over the selected region of the target surface. By adjusting the flow ratio of the guidance streams, the position of the focused liquid stream may be controlled. Thus, cells immobilized on the target surface may be selectively exposed to the focused liquid stream. While this method provides great accuracy with respect to positioning the hydrodynamically focused liquid, the method requires independent control over the flow rate of each stream. As the number of streams is increased, a relatively sophisticated and expensive flow control system is needed to ensure accuracy and repeatable stream positioning. Similarly, the methods and devices described in: U.S. Serial No. 60/286,819 (“A Method for Interacting a Product Substance with a Substance Retained on a Surface”), inventors Beyer, Krühne and Ahl; U.S. Serial No. 60/285,494 (“Sample Introduction into Apparatus for Hydrodynamically Focused Flow”), inventors Beyer and Krühne; U.S. Serial No. 60/286,550 (“Methods for Directing a Hydrodynamically Focused Flow of Liquid over a Topologically Variable Surface”), inventors Beyer, Krühne and Bonde; and U.S. Pat. No 6,200,814 to Malmqvist et al. suffer from the same drawback.

It has long been known that laminar flow may be employed to position samples that contain cells or small particles. For example, Tashiro et al. (2000), “Design and Simulation of Particles and Biomolecules Handling Micro Flow Cells with Three-Dimensional Sheath Flow,” Micro Total Analysis Systems 2000, pp. 209-212, describes a microfluidic device that employs a three-dimension sheath flow. In addition, a number of papers describe the use of laminar flow in assays. For example, Weigl et al. (1999), “Microfluid Diffusion-Based Separation and Detection,” Science 283(5400):346-347, describes a T-Sensor that combines separation and detection functions in a device that employs a sample solution, an indicator solution and a reference solution in parallel flow in a common channel. See also U.S. Pat. No. 5,716,852 to Yager et al. Similarly, Takayama et al. (1999) PNAS 96(10):5545-5548 describes a method to produce parallel streams of different liquids in a main channel to interact with a cell culture. The method was employed in Takayama et al. (2001), “Laminar Flows: Subcellular Positioning of Small Molecules,” Nature 411:1016, to selectively label a subpopulation of mitochondria in different regions of a cell. All of these papers describe the use of tributary channels that are coplanar to the main channel in order to introduce sample streams into the main channel. The coplanar configuration poses fabrication challenges when the tributary channels are closely spaced. Since the tributary channels are often connected via capillaries to fluid sources, the proximity of the tributary channels to each other makes it difficult to manipulate the individual connections. In addition, such a configuration is associated with an increased footprint. Thus, this is a suboptimal configuration for compact devices.

Thus, there is a need for low-cost, high-throughput and compact devices and methods that controllably expose a candidate compound to a live cell, thereby allowing analysis of candidate compound/live cell interaction without geometric constraints of prior art devices.

### SUMMARY OF THE INVENTION

Accordingly, it is an object of the present invention to overcome the above-mentioned disadvantages of the prior art by providing simplified devices and methods that effect controlled delivery of a fluid to a target region of a substrate surface. Such devices and methods may be employed to provide controlled exposure of a fluid containing a candidate compound to a live cell immobilized on the target region of the substrate surface.

Additional objects, advantages, and novel features of the invention will be set forth in part in the description that follows, and in part will become apparent to those skilled in the art upon examination of the following, or may be learned through routine experimentation upon practice of the invention.

In one embodiment, an inventive device is provided for exposing a portion of a target region of a substrate surface to a lane of reagent. The device includes a flow passage defined at least in part by a substrate having a target region on a surface thereof. Optionally, a cover plate and opposing side walls may define the flow passage. Also provided is a means for maintaining a carrier fluid in contiguous laminar flow through the flow passage and over the target region. Additionally, the device comprises an inlet for introducing a stream containing a reagent into carrier fluid that is upstream from the target region. When a reagent stream is introduced into the flow passage, a lane of reagent is formed downstream from the inlet over a portion of the target region, thereby exposing that portion of the target region to the reagent.

This embodiment may be employed to carry out an inventive method for exposing a portion of a target region of a substrate surface to a lane of reagent, comprising: (a) placing a flow passage defined at least in part by a substrate having a target region on a surface thereof; (b) maintaining a carrier fluid in contiguous laminar flow through the flow passage and over the target region; and (c) introducing a stream containing a reagent through an inlet into a carrier fluid upstream from the target region, thereby forming a lane of reagent downstream from the inlet over a portion of the target region.

In another embodiment, the invention relates to a device for exposing a target region of a substrate surface to a plurality of fluid lanes. The device comprises a flow passage defined at least in part by a substrate having a target region on a surface thereof. A plurality of inlets is provided in communication with the flow passage. Each of the inlets permits the introduction of a fluid in contiguous laminar flow through the flow passage to form a fluid lane downstream from the inlet over a portion of the target region. At least one of the inlets allow for fluid to be introduced directly into the flow passage from in a direction nonparallel to the flow passage. Preferably, the direction is substantially orthogonal to the flow passage. This device may be employed to carry out an inventive method for exposing a target region of a substrate surface to a plurality of fluid lanes, comprising: (a) placement of a flow passage defined at least in part by a substrate having a target region on a surface thereof; and (b) maintaining a plurality of fluids, each in contiguous laminar flow through the flow passage and each forming a fluid lane downstream from the inlet over a portion of the target region.

Typically, the inventive device and method may be employed to determine the effect of a plurality of candidate compounds on a monolayer of immobilized cells. This determination may be carried out through an inventive method, comprising: (a) immobilizing cells on a target region of a substrate surface; (b) placing the cells within a flow passage defined at least in part by the substrate surface; and (c) introducing a plurality of fluids, each in contiguous laminar flow through the flow passage and each forming a fluid lane containing a candidate compound downstream from the inlet over a portion of the target region.

### BRIEF DESCRIPTION OF THE DRAWINGS

FIGS. 1A-1C, collectively referred to as FIG. 1, illustrate an embodiment of the inventive device for exposing a portion of a target region of a substrate to a single lane of reagent. FIG. 1A illustrates the device in exploded view. FIG. 1B illustrates the device in schematic top view, before a stream containing a reagent is introduced therein. FIG. 1C illustrates in schematic view the device in operation, wherein a lane of reagent is formed over a portion of the target region.

FIG. 2 illustrates an embodiment of the inventive device similar to that illustrated in FIG. 1, except that a plurality of inlets are employed to form a plurality of reagent lanes.

FIGS. 3A-3B, collectively referred to as FIG. 3, illustrate another embodiment of the inventive device for exposing a portion of a target region of a substrate to a plurality of fluid lanes. FIG. 3A illustrates the device in exploded view. FIG. 3B illustrates the device in schematic top view in operation, wherein a plurality of reagent lanes is formed over the target region.

### DETAILED DESCRIPTION OF THE INVENTION

Before the invention is described in detail, it is to be understood that, unless otherwise indicated, this invention is not limited to particular materials, components, or manufacturing processes, as such may vary. It is also to be understood that the terminology used herein is for purposes of describing particular embodiments only, and is not intended to be limiting.

It must be noted that, as used in the specification and the appended claims, the singular forms “a,” “an,” and “the” include plural referents unless the context clearly dictates otherwise. Thus, for example, reference to “a lane” includes a single lane as well as a plurality of lanes, reference to “a reagent” includes a single reagent as well as a combination or mixture of reagents, reference to “an inlet” includes a single inlet as well as two or more inlets, and the like.

In this specification and in the claims that follow, reference will be made to a number of terms that shall be defined to have the following meanings:

The term “array” as used herein refers to a two-dimensional arrangement of features, such as cells or molecular moieties on a substrate surface. Arrays are generally comprised of regular, ordered features, as in, for example, a rectilinear grid, parallel stripes, spirals, lanes, and the like; but non-ordered arrays may be advantageously used as well. An array differs from a pattern in that patterns do not necessarily contain regular and ordered features.

The term “cell line” as used herein refers to a permanently established cell culture that will proliferate indefinitely if given appropriate fresh medium and space. While cell lines are readily available for some species, such as those in the rodent family, and difficult to establish for other species, such as humans, the term “cell line” as used herein is not limited to any particular species or cell type.

The term “expose” as to “expose a surface to a reagent” is used in its ordinary sense and refers to subjecting an item, e.g., a surface, or allow the item to be subjected to the influence of another item, e.g., a reagent, preferably via contact but optional through mere proximity. The items “exposed” to each other may or may not interact. [NEW]

The term “fluid-tight” is used herein to describe the spatial relationship between two solid surfaces in physical contact, such that fluid is prevented from flowing into the interface between the surfaces.

The term “laminar flow” as used herein refers to fluid movement in the absence of turbulence, such that mixing of fluid components occurs solely or primarily as a result of diffusion. The Reynolds number associated with laminar flow as described herein is typically about 0.01 to about 200, preferably about 0.01 to 20, and optimally about 0.1 to 20.

The term “lane” as used herein refers to one of a set of typically parallel and linear routes or courses along which a fluid travels or moves. While a lane may be bounded by one or more solid surfaces, a lane of fluid is bounded by at least one other fluid. “Optional” or “optionally” as used herein means that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “primary cell” is used herein in its ordinary sense and refers to a cell taken directly from a living organism that has not been immortalized.

The term “reagent” is used herein to refer to any substance used in a chemical, biochemical, or biological reaction to detect, measure, examine, or produce other substances.

The term “substrate” as used herein refers to any material having a surface over which laminar fluid flow may occur. The substrate may be constructed in any of a number of possible forms, such as wafers, slides, well plates, and membranes. Suitable substrate materials include, but are not limited to, supports that are typically used for cell handling, such as polymeric materials (e.g., polystyrene, polyvinyl acetate, polyvinyl chloride, polyvinyl fluoride, polyacrylonitrile, polyacrylamide, polymethyl methacrylate, polytetrafluoroethylene, polyethylene, polypropylene, polybutylene, polyvinylidene fluoride, polycarbonate, polyimide, and polyethylene teraphthalate), silica and silica-based materials, functionalized glasses, ceramics, and such substrates treated with surface coatings, polymeric and/or metallic compounds, or the like. While the foregoing support materials are representative of conventionally used substrates, it is to be understood that the substrate may in fact comprise any biological, nonbiological, organic and/or inorganic material, and may further have any desired shape, such as a disc, square, sphere, circle, etc. The substrate surface is typically but not necessarily flat; e.g., the surface may contain raised or depressed regions. In addition, while the substrate may exhibit autofluorescence, it preferable that autofluorescence is minimized or avoided.

The term “surface modification” as used herein refers to the chemical and/or physical alteration of a surface by an additive or subtractive process that changes one or more chemical and/or physical properties of a substrate surface, or a selected location or region of a substrate surface. For example, surface modification may involve (1) changing the wetting properties of a surface; (2) functionalizing a surface, i.e., providing, modifying, or substituting surface functional groups; (3) defunctionalizing a surface, i.e., removing surface functional groups; (4) otherwise altering the chemical composition of a surface, e.g., through etching; (5) increasing or decreasing surface roughness; (6) providing a coating on a surface, e.g., a coating that exhibits wetting properties that are different from the wetting properties of the surface; and/or (7) depositing particulates on a surface.

The term “target region” as used herein refers to a predefined two-dimensional area over which fluid is directed to flow. The target region is typically but not necessarily contiguous and may or may not have cells adhered thereto.

Thus, the invention generally relates to a device for exposing a portion of a target region of a substrate surface to a lane of reagent. The device comprises a flow passage defined at least in part by a substrate having a target region on a surface thereof, an optional cover plate having a surface that opposes the surface of the substrate, and optional opposing side walls in fluid-tight contact with the substrate. A carrier fluid maintaining means is provided for maintaining a carrier fluid in contiguous laminar flow through the flow passage and over the target region. An inlet is provided in communication with the flow passage for introducing a stream containing a reagent into a carrier fluid upstream from the target region. As a result, a lane of reagent is formed downstream from the inlet over a portion of the target region. Laminar flow ensures that the reagent is exposed only to a selected portion of the target region. Thus, when appropriate proper fluids and reagents are selected, the device may be employed to carry out substrate surface modification. When live cells are immobilized the target region, the inventive device provides a simple and inexpensive means for accurate delivery of candidate compounds to selected cells. Both surface modification and candidate compound delivery may be achieved by the invention without need for complex flow control mechanisms, such as those found in devices that employ guide streams to hydrodynamically focus and position a reagent stream. As a result, the invention provides a novel approach to surface modification, as well as a means for rapid and information-rich analysis of candidate compound/live cell interaction.

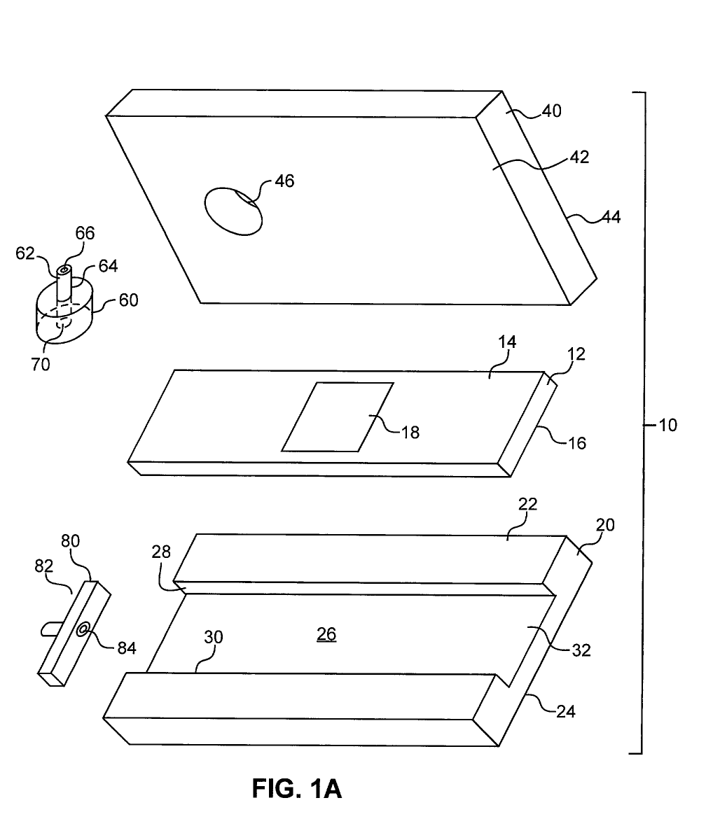

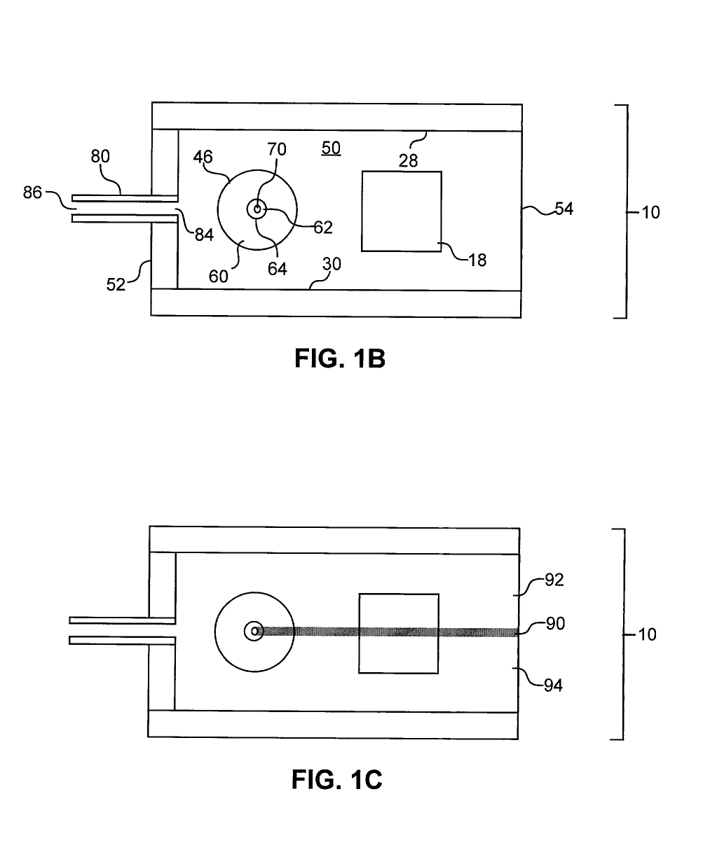

FIG. 1 illustrates an embodiment of the inventive device. As with all figures referenced herein, in which like parts are referenced by like numerals, FIG. 1 is not necessarily to scale, and certain dimensions may be exaggerated for clarity of presentation. The device 10 includes a substrate 12 comprising first and second substantially planar opposing surfaces indicated at 14 and 16, respectively, and is comprised of a material that is substantially inert with respect to the fluids that will be transported through the device. The surfaces 14 and 16 are rectangular in shape and parallel to each other. While FIG. 1 illustrates that a square-shaped target region 18 is located at the center of surface 14, the target region may be of any size (or shape) as long as it is no larger than surface 14. For square shaped target regions, the surface area of the target region is typically 1 mm² to about 100 mm², preferably about 10 mm² to about 50 mm², and optimally about 20 mm² to about 30 mm².

The inventive device also includes an optional base 20 having opposing surfaces indicated at 22 and 24, respectively. A channel 26 located on the first surface 22 is defined by parallel opposing side walls 28 and 30, and by the floor 32, which extends along the length of the base 20. The channel is sized and shaped to snugly contain the substrate 12, such that fluid-tight contact can be established between the substrate 12 and side walls 28 and 30. It will be readily appreciated that, although the channel 26 has been represented in a generally extended form to correspond to the shape of the rectangular substrate 12, channels for this and other embodiments can have a variety of configurations depending on the shape of the substrate. The channel 26 has a carrier inlet terminus 34 at a first end and an outlet terminus 36 at the opposing end. As shown in FIG. 1, both termini 34 and 36 are located at opposing edges of the base surface 22.

The device 10 also includes an optional cover plate 40 that is complementarily shaped with respect to the base 20 and has first and second substantially planar opposing surfaces indicated at 42 and 44, respectively. The contact surface 42 of the cover plate 40 is typically capable of interfacing closely with the contact surface 22 of the base 20 to achieve fluid-tight contact between the surfaces. Alternatively, a gasket material may interposed between the contact surfaces 42 and 44. The cover plate 40 may be substantially immobilized over the target region 18, and the cover plate contact surface 42 in combination with the upper surface 14 of the substrate and with the side walls 28 and 30 of the channel 26 may define a flow passage 50 through which a carrier fluid may flow. That is, the cover plate 40 serves as a roof and the substrate 12 serves as the floor of the flow passage 50. Located at the upstream end of the flow passage is a carrier fluid opening 52, and an outlet 54 is located at the downstream end of the flow passage. When the contact surfaces of the cover plate and the substrate are in fluid-tight contact, the flow passage is fluid-tight as well. The cover plate 40 can be formed from any suitable material for forming the substrate 12. To ensure that the flow passage is fluid-tight, pressure-sealing techniques may be employed, e.g., by using external means to urge the pieces together (such as clips, tension springs, or an associated clamp). Additionally or alternatively, the base and the cover plate may be held together through appropriate application of a vacuum. As with all embodiments described herein, the sealing techniques may allow the contact surfaces of the cover plate and the base to remain in fluid-tight contact under a pressure associated with laminar fluid flow, i.e., an internal device fluid pressure of up about 5 bars, typically about 2 bars to about 5 bars, optimally about 2 bars.

As shown in FIG. 1A, the cover plate, substrate 12, and the base may each be discrete components. In such a case, alignment means such as a plurality of appropriately arranged protrusions in component parts, e.g., projections, depressions, grooves, ridges, guides, or the like, known to one of ordinary skill in the art, may be employed to align the cover plate with the base. In some instances, however, the substrate and the cover plate may be attached to each other. For example, the cover plate and the base may be hinged together to provide repeatable contact between the contact surfaces thereof. In such a case, the hinge also serves as an alignment means.

The cover plate may include a variety of features. As shown, a circular opening 46 is provided extending through the cover plate 40 in a direction orthogonal to cover plate surfaces 42 and 44, so as to allow communication therebetween. The opening 46 is located between the carrier flow inlet 52 and the target region 18, approximately at the midpoint between the side walls 28 and 30 of the flow passage 50. The opening 46 may be plugged by a septum 60 of an elastic material, such as butyl or silicone rubber, which is capable of providing a fluid-tight seal against the surface that defines the opening. Extending through the septum 60 and the opening is an introduction tube 62 having an end in communication with flow passage, the end representing an inlet 70 for introducing a reagent into the flow passage. That is, the septum 60 encircles the exterior surface 64 of the introduction tube 62. Another end 66 of the introduction tube 62 may communicate fluidly with a source of reagent (not shown).

The septum 60 also provides a fluid-tight seal against the introduction tube 62 to ensure that fluid cannot travel into or out of the flow passage 50 through the interface between the septum and the introduction tube. Optionally, the septum is made from a self-sealing material such that no opening remains if the introduction tube is withdrawn from the septum. Preferably, portions of the septum 60 and the introduction tube 62 that are exposed to flow passage 50 lie substantially flush with the contact surface 42 of the cover plate.

An additional septum 80 may provide a fluid-tight seal against the interior surface of the flow passage 50 at the carrier fluid opening 52 to allow carrier fluid tube 82 to communicate though carrier fluid inlet 84 with the flow passage 50 as well. Another end 86 of the carrier tube may provide fluid communication with a source of carrier fluid (not shown).

Thus, as illustrated in FIG. 1B, the device is assembled to form a flow passage 50 defined by the substrate in the base, the side walls 28 and 30, and the cover plate. The carrier fluid is introduced through the carrier fluid inlet 84 and maintained in contiguous laminar flow through the flow passage 50, over the target region 18 and through the outlet. Preferably, the carrier fluid is maintained in contiguous laminar flow at a constant volumetric flow rate and velocity. Constant flow rate may be achieved through a number of means discussed below.

As a result, the carrier fluid fills the entire flow passage 50 and flows over and covers the entire target region 18. Then, as illustrated in FIG. 1C, a stream containing a reagent is introduced through inlet 70 of the introduction tube 62 and into the carrier fluid while the carrier fluid is still flowing though the flow passage 50. Preferably, the stream is introduced at a laminar flow rate to ensure that nondiffusional mixing does not occur. As a result, the carrier fluid conveys the stream of reagent toward the outlet 54 of the device, thereby forming a lane 90 of reagent downstream from the inlet 70 and over a portion of the target region 18. The lane 90 of reagent is bounded above by the cover plate and below by the substrate. However, carrier fluid lanes 92 and 94 define the side boundaries of the reagent lane. As shown, the lane passes over a portion of the target region 18 interposed between two regions that have been previously exposed to only the carrier fluid. Accordingly, the portion of the target region under the lane is exposed to the reagent. If the flow of reagent is stopped before the flow of carrier fluid is discontinued, the carrier fluid may again flow over the entire target region.

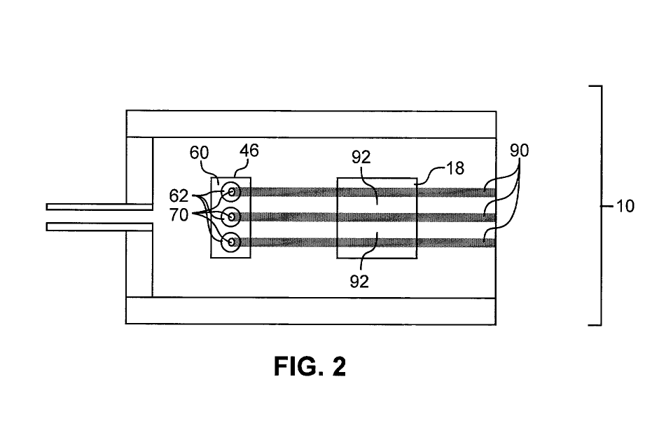

Alternatively, the above embodiment may be adapted to form a plurality of reagent lanes over the target region. FIG. 2 illustrates a device 10 identical to that illustrated in FIG. 1 except that the opening 46 through the cover plate is rectangular in shape and has a length approximately that of the length of the square target region. The rectangular opening 46 is plugged by a septum 60 having a plurality of introduction tubes, each indicated at 62 extending therethrough, and each having an end in communication with flow passage. Located at each end is an inlet 70 for introducing a reagent into the flow passage. The tubes are aligned along the length of the rectangular septum 60. This embodiment allows a plurality of streams, each containing a reagent, to be introduced into the carrier fluid to form lanes of reagent downstream from the inlets 70 and over a portion of the target region 18. Preferably, lanes of the carrier fluid separate the lanes 92 of reagent. This adapted embodiment allows portions of the target region 92 to be exposed to parallel lanes of reagent simultaneously when flow reagents occur simultaneously through the inlets 70.

Another embodiment of the invention relates to a device for exposing a target region of a substrate surface to a plurality of fluid lanes. As before, this embodiment comprises a flow passage defined at least in part by a substrate having target region on a surface thereof. No carrier means is needed for maintaining a carrier fluid in contiguous laminar flow through the flow passage and over the target region. However, a plurality of inlets is provided, each for introducing a fluid in contiguous laminar flow through the flow passage to form a fluid lane downstream from the inlet over a portion of the target region. At least one of the inlets allows fluid to be introduced directly into the flow passage in a direction noncoplanar to the substrate surface. The laminar flow ensures that the fluids do not mix and that each fluid lane is exposed only to a selected portion of the target region. This device allows a plurality of reagent lanes to flow over the target region if at least two fluids each contain a reagent.

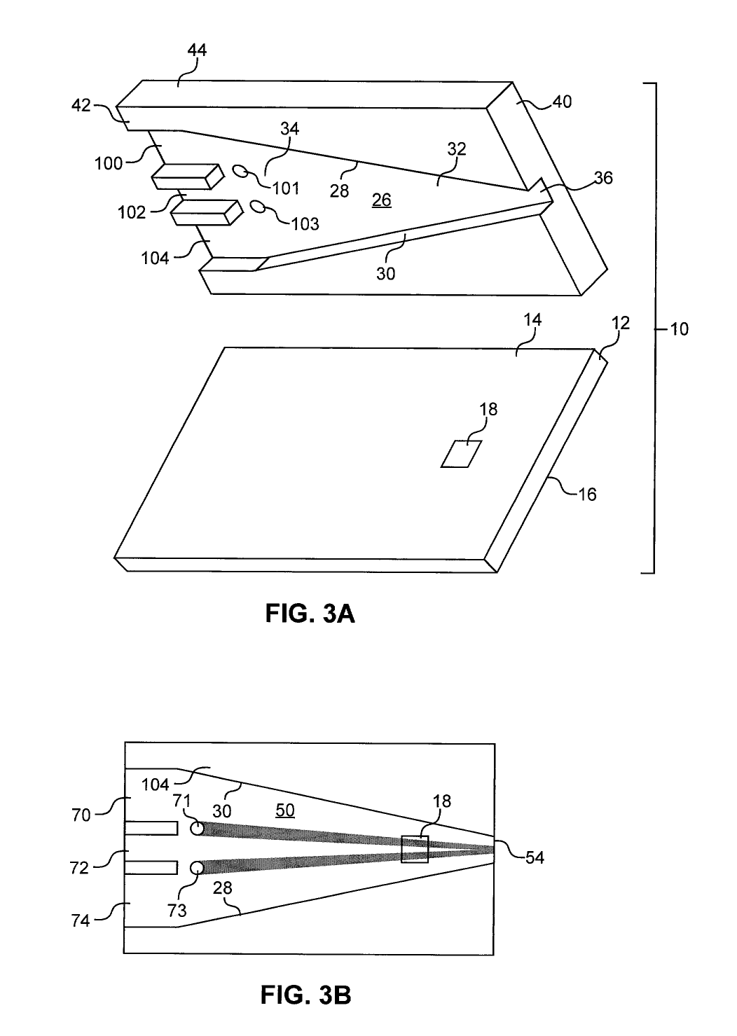

FIG. 3 illustrates an example of this embodiment of the inventive device. As illustrated in FIG. 3A, the device 10 includes a substrate 12 comprising first and second substantially planar opposing surfaces indicated at 14 and 16, respectively, wherein the target region 18 is located at the center of surface 14. The device 10 also includes an optional cover plate 40 having first and second substantially planar opposing surfaces indicated at 42 and 44, respectively. A main channel 26 located on the first surface 42 is defined by opposing side walls 28 and 30, and ceiling 32 extending along the length of the cover plate 20. The main channel 26 has an inlet terminus 34 at a first end and an outlet terminus 36 at the opposing end. As shown in FIG. 3A, terminus 34 is located away from the exterior edges of the first cover plate surface 42, while terminus 36 is located on the edge of the first cover plate surface 42. As shown, the distance between side walls 28 and 30 decreases from the inlet terminus 34 to the outlet terminus 36, though this is not a requirement. A plurality of introduction channels, indicated at 100, 102 and 104 extend parallel to each other from the exterior edge opposing the main channel outlet terminus 36 to the inlet terminus 34. Openings 101 and 103 extend through the cover plate 40 and are located between and slightly downstream from the introduction channels.

The contact surface 42 of the cover plate 40 is typically capable of interfacing closely with the contact surface 14 of the substrate 12 to achieve fluid-tight contact between the surfaces. The cover plate 40 may be substantially immobilized over the substrate contact surface 14. As a result, the substrate contact surface 14 in combination with the ceiling 32 and the side walls 28 and 30 of the channel 26 defines a main flow passage 50 through which fluids may flow. Similarly, the substrate contact surface 14, in combination with introduction channels 100, 102 and 104 form introduction conduits, each having an inlet indicated at 70, 72 and 74 through which fluid external to the microdevice may flow, emptying into the main flow passage 50. In addition, openings 101 and 103 form inlets indicated at 71 and 73, respectively. Inlets 71, 73 allow fluid to be introduced directly into the flow passage 50 in a direction noncoplanar to the substrate surface. In this instance, case, the direction of fluid introduced directly into the flow passage is orthogonal the plane defined by the flow passage 50. Outlet 54 is located at the downstream end of the flow passage. When the contact surfaces of the cover plate and the substrate are in fluid-tight contact, the main flow passage and the introduction conduits are fluid-tight as well. Pressure-sealing techniques may be employed to ensure that the flow passage remains fluid-tight. The introduction conduits typically provide fluid communication with a plurality of fluid sources.

In operation, as illustrated in FIG. 3B, the device is assembled to form the main flow passage 50 defined by the substrate, the side walls 28 and 30, and the ceiling of the main channel. The target region 18 is located within the main flow passage 50 downstream from the inlets 70, 71, 72, 73, and 74. These inlets each provide fluid communication with a fluid source from which fluid flows, making it possible to maintain the fluids in contiguous laminar flow through the flow passage to form fluid lanes 90 and 92, extending from each of the inlets 70, 71, 72, 73, and 74 over a portion of the target region 18. Since the main flow passage 50 narrows from the inlets 70, 71, 72, 73, and 74 to the outlet 54, the formed lanes 90 and 92 also narrow downstream.

The specific geometry of the device components plays an important role in controlling the accuracy and precision of the fluid lane placement. Thus, while the substrate is the only necessary component that provides a surface to define the flow passage, it is preferred that the flow passage be further defined by other components as well. As discussed above, the flow passage is typically defined in part by a cover plate positioned over the target region of the substrate surface. Similarly, it is preferred that the flow passage of the device be constructed as a conduit. Accordingly, the flow passage is typically defined not only by the cover plate and the substrate surface, but also by opposing side walls in fluid-tight contact with the substrate. In some instances, the side walls represent an integral portion of the substrate. When the flow passage is a conduit having a constant cross-sectional shape and area, formed lanes are substantially parallel to each other and to the conduit walls. Conversely, the width of the lanes may vary according to variations in the cross-sectional shape of the conduit. Lanes may be narrowed if the conduit is narrowed as well. Thus, for example, when the distance between the side walls decreases along the flow passage downstream from the inlet, the lanes of fluid flowing over the target region will be narrowed as well. This phenomenon may be exploited to ensure that lanes of an appropriate width flow over the target region. By employing appropriate geometries and flow rates, lanes having a width of a few micrometers or less can be formed. However, without side walls and/or a cover plate, the flow rates may be adjusted to compensate for the fluid constraining forces provided by these solid components.

Similarly, the cover plate and substrate surfaces may or may not be parallel to each other. Because the reagents and fluids to be employed with the invention are often rare or expensive, it is preferred that as little reagent and fluid be used as practicable to flow over the target region. However, fluid flow depends on the volume of reagent or fluid as well as the volume of the flow passage. Typically, when the substrate and cover plate surfaces are parallel to each other, the surfaces are located from about 1 μm to about 500 μm from each other. Preferably, the substrate and cover plate surfaces are located from about 20 μm to about 100 μm from each other.

For any of the embodiments described above, it is preferred that the device be constructed in a modular manner to ensure interchangeability of the components. In particular, stock items can be used to form certain components, thereby lowering the overall cost of the device and rendering it feasible, if desired, to dispose of the stock items after use. For example, the substrate may consist of an ordinary 25 mm×75 mm or 50 mm×75 mm glass slide, an item found in most laboratories. Similarly, to facilitate handling, the components of the inventive device may be detachable from one another. As access to the target region of the substrate is limited when it is in an opposing relationship to the cover plate, it is preferred that the substrate be detachable from the cover plate. Using a detachable and disposable item as the substrate, such as a glass slide, avoids the complex capillary tube attachment procedures before each use of the device that are required when the tubes are essentially permanently connected to the inlets.

Typically, at least one inlet is required for each reagent used in conjunction with the device. When a plurality of reagents is employed, the inlets may be positioned such that the lanes of reagents do not contact each other. For the device illustrated in FIG. 2, the inlets may be positioned such that the lanes of reagent are separated by lanes of carrier fluid. To achieve reagent lane separation for the device illustrated in FIG. 3, alternating inlets may each fluidly communicate with the same source of fluid. Thus, the device illustrated in FIG. 3 is utilized to form lanes of different reagents separated by lanes of the same inert fluid: one set of alternating inlets 70, 72, and 74 fluidly communicates with a source of carrier fluid, and the other set of alternating inlets 71 and 73 fluidly communicates with a different source of reagent. In some instances, though, lanes of different reagents may be positioned adjacent to each other.

It is envisioned that the inventive device may be constructed to handle any number of reagents. Commercially available fluid handling apparatuses, e.g., autosamplers and microtiter plates, may handle a fixed number fluids, and the inventive device may be constructed to interface with these apparatuses. As such, apparatuses are ordinarily constructed to handle 8,96,384, or 1536 different fluids. Thus, the device may include a corresponding number of inlets as well.

The device may be adapted to form lanes from fluids of virtually any type and amount desired, depending on the intended purpose for lane formation. For example, when it is desirable for to etch channels of a particular width in each substrate surface, lanes containing acid as a reagent and having a width corresponding to the desired width of the channels may flow over the target region. Thus, the fluid may be aqueous and/or nonaqueous. Nonaqueous fluids include, for example, organic solvents, and lipidic liquids. When the invention is employed to carry out cellular assays, as described below, typical reagents include but are not limited to, pharmacologically active agents and stains. Each reagent lane may contain the same reagent, optionally at different concentrations. In addition or optionally, each lane may contain a different reagent.

Whether fluid flow is laminar depends on several variables, such as: the geometry of the surfaces over which the fluid flows, flow velocity, and fluid properties such as viscosity. It is thus important that fluid movement in the inventive device be precisely controlled to maintain laminar flow. As components of this control, inlets through which fluids containing reagents are introduced into the flow passage typically have a cross sectional area of 1×10⁻⁵ mm² to about 1 mm², preferably about 5×10⁻⁴ to about 0.1 mm², and optimally 1×10 mm³ to about 1×10⁻² mm². The inlets may have a variety of shapes including, but not limited to, circular, oval, square, rectangular, and triangular.

In order to ensure that laminar flow is exhibited in the lanes formed downstream from the carrier liquid, a pump is employed to deliver appropriate fluid from a fluid source through the appropriate inlet. Typically, high precision microsyringe pumps are employed to provide fluid flow through capillaries to the inlets. Other types of pumps, however, may be employed. In some instances, one pump is sufficient to provide a motive force to ensure proper fluid flow. That is, each inlet may fluidly communicate with a source of reagent that is pressurized by the same pressure generating means. In other instances, however, each inlet may fluidly communicate with an independently controlled pressure generating means. While independent control of fluid introduction into the flow path typically involves added cost, such control allows for nonsimultaneous formation of lanes. Thus, selected portions of the target region may be exposed to reagents for differing periods. For example, if each of a plurality of inlets is adapted to allow through transport of the same reagent-containing fluid, independent control allows different portions of the target region to be exposed to the same reagent for different periods. This allows for the systematic study of the effect of a reagent on a target region as a function of time.

The inventive device described herein can be adapted for use in connection with a cell-based assay. Cell-based assays represent an important means for determining the effects of reagents on cells, particularly living cells. For example, a potential new drug can be assayed against an intact and living cell in the present method, thereby providing improved pharmacodynamic and pharmacokinetic modeling over conventional assays that incorporate nonliving cells, or molecular assays, e.g., affinity assays.

Thus, the invention additionally provides a method for screening cells with respect to a selected reagent as well as a method for selectively exposing a cell to a reagent. Both methods involve immobilizing a cell on a portion of the target region of the substrate such that the cell is downstream from the inlet associated with the selected reagent, and allowing a stream containing the reagent to form a lane flowing over the immobilized cell, thereby allowing the reagent to contact the cell. For screening, the method further comprises determining whether the cell has changed, e.g., in morphology, or whether the cell has caused a change to the fluid, the reagent, or another substance in the fluid, e.g., expressed a protein into the lane as an indicator of the biological activity of the reagent toward the cell.

Preferably, either the carrier fluid or the fluid in at least one of the formed lanes comprises a culture medium for sustaining the viability of the cell. It must be noted, however, that the culture medium does not necessarily ensure that the cell remains living, although living cells are preferred. Thus, for example, the culture medium may be provided to keep living cells viable in the absence of a toxic reagent. If a toxic reagent is introduced into the flow cell, e.g., during a toxicity study, cell death may result notwithstanding the presence of the culture medium.

Culture media suitable for any particular cell will be known to those skilled in the art and are available commercially from, for example, Sigma Inc., St. Louis, Mo.

Generally, such media contain mixtures of salts, amino acids, vitamins, nutrients, and other substances necessary to maintain cell health. Preferred salts in the culture medium include, without limitation, NaCl, KCl, NaH₂PO₄, NaHCO₃, CaCl₂, MgCl₂ and combinations thereof. Preferred amino acids are the naturally occurring L amino acids, particularly arginine, cysteine, glutamine, histidine, isoleucine, leucine, lysine, methionine, phenylalanine, threonine, tryptophan, tyrosine, valine, and combinations thereof. Preferred vitamins in the cell culture include, for example, biotin, choline, folate, nicotinamide, pantothenate, pyridoxal, thiamine, riboflavin and combinations thereof. Glucose and/or serum, e.g., horse serum or calf serum, are also preferred components of the culture medium. Optionally, antibiotic agents such as penicillin and streptomycin may be added to suppress the growth of bacteria. Preferably, the culture medium will contain one or more protein growth factors specific to a particular cell type. For example, many nerve cells require trace amounts of nerve growth factor (NGF) to sustain their viability. Similarly, the culture medium will preferably contain hepatocyte growth factor (HGF) when hepatocytes are present in the assay. Those skilled in the art routinely consider these and other factors in determining a suitable culture medium for any given cell type. The culture medium can be present in one or both of the guide streams and optionally in the fluid stream containing the reagent.

Nearly any type of cell may be used with the present methods, including both eukaryotic cells and prokaryotic cells. Preferably, however, the cell is a primary cell obtained from a mammal, e.g., a human. Preferred cell types are selected from the group consisting of blood cells, stem cells, endothelial cells, epithelial cells, bone cells, liver cells, smooth muscle cells, striated muscle cells, cardiac muscle cells, gastrointestinal cells, nerve cells, and cancer cells. Alternatively, the immobilized cells may originate from a cell line.

The substrate surface on which the target region is located may be selected for facile immobilization of cells. Such solid surfaces include, for example, a collagen-derivatized surface, dextran, polyacrylamide, nylon, polystyrene, and combinations thereof. Typically, immobilized cells are present on the target region as a monolayer. The monolayer may be substantially contiguous or comprise an array of features, each feature comprising at least one cell. All or substantially all of the immobilized cells may be of the same type. The monolayer may be immobilized on the solid surface using conventional techniques known to those skilled in the art. For example, the cells may be immobilized on the target region by simply contacting the target region with the cells. Optionally, a centrifuge may be used. Generally, the force required to immobilize a cell on the target region is from about 200×g to about 500×g.

Alternatively, the surface may be coated with a layer of a cell-adhering substance, such as collagen, alginate, agar, or other material to immobilize the cells. When immobilization of cells in a contiguous layer is desired, the cell-adhering substance may be contiguously coated on the target region. When it is desirable to provide an immobilized array of cells, however, the cell-adhering substance may be present as an array of features on the target region. That is, an array of locations on the target region may be coated with an appropriate material to form an array, e.g., patterns such as lanes, checkerboards, spots or others, so that cells may be spatially arranged at specific locations on the solid surface. See, e.g., U.S. Pat. Nos. 5,976,826 and 5,776,748 to Singhvi.

Alternatively, the cells may be present on the target region as a tissue sample. Immobilization of tissue samples containing cells of interest may be accomplished by first freezing, e.g., to about −15° C. to about −20° C., a relatively large section of tissue. Thereafter, a knife, microtome, or similar sectioning device is used to slice the frozen tissues into sections. Next, a single section of the tissue is placed onto the target region, e.g., a glass slide, and the section is allowed to “melt” on the target region, thereby immobilizing the cells in the tissue on the target region. Those skilled in the art will recognize other immobilization techniques that can be used.

Once the cell or tissue containing the cells of interest is immobilized, a stream of fluid containing the reagent is generated to form a lane that contacts the cell or cells of interest. In this way, the reagent is placed in contact with the cell or cells of interest. If the cells are immobilized in an array, the array features and the fluid lanes must be appropriately aligned to effect precise and accurate delivery of the reagent to the cells of interest.

As stated above, the present method provides a method for screening the biological activity of a reagent with respect to a particular cell type. Biological activity of the reagent can be detected by determining whether the cell changes in response to the reagent, for example, by changing its shape or expressing a protein. Generally, a means for visually observing or otherwise detecting such changes is used. Such means include, for example, use of a microscope, chromatographic methods, an immunoassay, a fluorescence detector, a radioactivity detector, and combinations thereof.

As will be appreciated, different assays require the detection of different types of biological activity. In some cases, determining the particular biological activity of a reagent can be accomplished by direct observation of the cell. For example, toxicity assays of a reagent may involve detecting cell death. An assay testing for mitotic activity of a reagent will detect the presence of new cells. In other assays, it is preferred to detect changes in the fluid or reagent that are caused by the cell. For example, determining biological activity may be accomplished by assaying outflow material to detect substances excreted by the cell in response to the reagent.

Thus, the cell-based assays described herein are useful for screening reagents, e.g., drug or drug candidates, for a number of biological activities. Examples of biological activities that can be screened include, without limitation, cellular differentiation, proliferation, locomotion, toxicity, apoptosis, adhesion, translocation of signaling molecules, protein expression, and oncogenic transformation. In addition, the present method allows for the ability to screen for adsorption, absorption, distribution, metabolism, and/or excretion properties of a reagent.

Thus, variations of the present invention will be apparent to those of ordinary skill in the art. For example, while a channel may be provided on a cover plate or base surface, as described above, the channels may be instead located on the substrate surface. In addition, the inventive device may be employed to carry out biomolecular assays by immobilizing biomolecules in place of cells on the target region. Furthermore, the inventive device may be used in combination with other reagent deposition techniques, such as the “spotting” techniques well known in the art for depositing arrays such as oligonucleotide arrays.

It is to be understood that while the invention has been described in conjunction with the preferred specific embodiments thereof, that the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

We claim:

1. A device for exposing a portion of a target region of a substrate surface to a lane of reagent, the device comprising:
a flow passage defined at least in part by a substrate having a target region on a surface thereof;
a means for maintaining a carrier fluid in contiguous laminar flow through the flow passage and over the target region; and
an inlet for introducing a stream containing a reagent into the carrier fluid upstream from the target region, thereby forming a lane of reagent downstream from the inlet over a portion of the target region and exposing the portion to the reagent.

2. The device of claim 1, wherein the flow passage is further defined by a cover plate having a surface that opposes the surface of the substrate.

3. The device of claim 2, wherein the substrate is detachable from the cover plate.

4. The device of claim 2, wherein the substrate surface is substantially planar.

5. The device of claim 2, wherein the substrate and cover plate surfaces are substantially parallel.

6. The device of claim 5, wherein the substrate and cover plate surfaces are located from about 1 μm to about 500 μm from each other.

7. The device of claim 6, wherein the opposing surfaces of the substrate and cover plate are located from about 20 μm to about 100 μm from each other.

8. The device of claim 1, wherein the flow passage is further defined by opposing side walls in fluid-tight contact with the substrate.

9. The device of claim 8, wherein the side walls are substantially parallel to each other and to the lane of reagent.

10. The device of claim 8, wherein the distance between the side walls decreases along the flow passage downstream from the inlet.

11. The device of claim 1, wherein the means for maintaining carrier fluid in contiguous laminar flow is adapted to provide for constant velocity flow.

12. The device of claim 1, wherein the inlet is positioned to introduce reagent into the flow passage through the cover plate.

13. The device of claim 1, further comprising at least one additional inlet for the introduction of at least one additional stream of reagent into the carrier fluid upstream from the target region, thereby forming a plurality of lanes of reagents downstream over different portions of the target region.

14. The device of claim 13, wherein the inlets are positioned such that the lanes of reagent are separated by lanes of carrier fluid.

15. The device of claim 13, wherein the inlets comprise 8 inlets.

16. The device of claim 15, wherein the inlets comprise 96 inlets.

17. The device of claim 13, wherein each inlet fluidly communicates with a source of reagent that is pressurized by the same pressure generating means.

18. The device of claim 13, wherein each inlet fluidly communicates with an independently controlled pressure generating means.

19. The device of claim 1, further comprising cells immobilized on the target region.

20. The device of claim 19, wherein the cells are present on the target region as a monolayer.

21. The device of claim 20, wherein the monolayer is substantially contiguous.

22. The device of claim 20, wherein the monolayer comprises an array of features, each feature comprising at least one cell.

23. The device of claim 20, wherein substantially all of the immobilized cells are of the same type.

24. The device of claim 19, wherein the cells are present on the target region as a tissue sample.

25. The device of claim 19, wherein the cells are primary cells.

26. The device of claim 19, wherein the cells are from a cell line.

27. The device of claim 1, further comprising a cell-adhering substance on at least a portion of the target region.

28. The device of claim 27, wherein the cell-adhering substance is contiguously coated on the target region.

29. The device of claim 27, wherein the cell-adhering substance is present as an array of features on the target region.

30. The device of claim 29, wherein the cell-adhering substance is a collagenic substance.

31. The device of claim 1, further comprising a plurality of biomolecules immobilized on the target region.

32. A method for exposing a portion of a target region of a substrate surface to a lane of reagent, comprising:
(a) providing a flow passage defined at least in part by a substrate having a target region on a surface thereof;
(b) maintaining a carrier fluid in contiguous laminar flow through the flow passage and over the target region; and
(c) introducing a stream containing a reagent through an inlet into the carrier fluid upstream from the target region, thereby forming a lane of reagent downstream from the inlet over a portion of the target region and exposing the portion to the reagent.

33. The method of claim 32, wherein the target region contains cells immobilized thereon.

34. The method of claim 33, wherein the carrier fluid is a medium appropriate to sustain living cells.

35. The method of claim 32, wherein the reagent is a pharmacologically active agent.

36. The method of claim 32, wherein the reagent is a stain.

37. The method of claim 32, further comprising: (d) introducing at least one additional stream of reagent into the carrier fluid upstream from the target region, thereby forming a plurality of lanes of reagents downstream over different portions of the target region.

38. The method of claim 37, wherein step (d) is carried out simultaneously with step (c).

39. The method of claim 37, wherein each stream contains the same reagent.

40. The method of claim 39, wherein each stream contains the same reagent at a different concentration.

41. The method of claim 37, wherein each stream contains a different reagent.

42. The method of claim 32, wherein step (c) is terminated before step (b).

43. The method of claim 32, further comprising: (d) inspecting the portion of the target region contacted by the lane of reagent.

44. A device for exposing a target region of a substrate surface to a plurality of fluid lanes, the device comprising:
a flow passage defined at least in part by a substrate having a target region on a surface thereof; and
a plurality of inlets, each for introducing a fluid in contiguous laminar flow through the flow passage to form a fluid lane downstream from the inlet over a portion of the target region, thereby exposing the portion to the fluid,
wherein at least one inlet is positioned to introduce fluid directly into the flow passage in a direction noncoplanar to the substrate surface.

45. The device of claim 44, wherein the traversing direction is substantially orthogonal to the substrate surface.

46. The device of claim 44, wherein the flow passage is further defined by a cover plate having a surface that opposes the surface of the substrate.

47. The device of claim 46, wherein the substrate is detachable from the cover plate.

48. The device of claim 46, wherein the substrate surface is substantially planar.

49. The device of claim 44, wherein the substrate and cover plate surfaces are substantially parallel.

50. The device of claim 49, wherein opposing surfaces of the substrate and cover plate are located from about 1 μm to about 500 μm from each other.

51. The device of claim 50, wherein the opposing surfaces of the substrate and cover plate are located from about 20 μm to about 100 μm from each other.

52. The device of claim 44, wherein the flow passage is further defined by opposing side walls in fluid-tight contact with the substrate.

53. The device of claim 52, wherein the side walls are substantially parallel to each other and to the lane of reagent.

54. The device of claim 52, wherein the distance between the side walls decreases along the flow passage downstream from the inlet.

55. The device of claim 44, wherein the inlets are positioned in a line perpendicular to the fluid lanes.

56. The device of claim 55, wherein alternating inlets fluidly communicate with a source of carrier fluid.

57. The device of claim 55, wherein alternating inlets each fluidly communicate with a different source of reagent.

58. The device of claim 44, wherein each inlet fluidly communicates with a source of fluid that is pressurized by the same pressure generating means.

59. The device of claim 44, wherein each inlet fluidly communicates with an independently controlled pressure generating means.

60. The device of claim 44, further comprising cells immobilized on the target region.

61. The device of claim 60, wherein the cells are present on the target region as a monolayer.

62. The device of claim 61, wherein the monolayer is substantially contiguous.

63. The device of claim 61, wherein the monolayer comprises an array of features, each feature comprising at least one cell.

64. The device of claim 61, wherein substantially all of the immobilized cells are the same type.

65. The device of claim 60, wherein the cells are present on the target region as a tissue sample.

66. The device of claim 60, wherein the cells are primary cells.

67. The device of claim 60, wherein the cells are from a cell line.

68. The device of claim 44, further comprising a cell-adhering substance on at least a portion of the target region.

69. The device of claim 68, wherein the cell-adhering substance is contiguously coated on the target region.

70. The device of claim 68, wherein the cell-adhering substance is present as an array of features on the target region.

71. The device of claim 68, wherein the cell-adhering substance is a collagenic substance.

72. The device of claim 44, further comprising a plurality of biomolecules immobilized on the target region.

73. A method for exposing a target region of a substrate surface to a plurality of fluid lanes, comprising:
(a) providing a flow passage defined at least in part by a substrate having a target region on a surface thereof; and
(b) maintaining a plurality of fluids, each in contiguous laminar flow through the flow passage, each fluid forming a fluid lane downstream from the inlet over a portion of the target region, thereby exposing the portion to the fluid,
wherein at least one fluid is introduced directly into the flow passage in a direction noncoplanar to the substrate surface.

74. The method of claim 73 wherein the traversing direction is substantially orthogonal to the flow passage.

75. The method of claim 73, wherein the target region contains cells immobilized thereon.

76. The method of claim 75, wherein at least one fluid comprises a medium appropriate to sustain living cells.

77. The method of claim 73, wherein at least one fluid comprises a reagent.

78. The method of claim 77, wherein the reagent is a pharmacologically active agent.

79. The method of claim 77, wherein the reagent is a stain.

80. The method of claim 77, further comprising: (c) inspecting the portion or portions of the target region contacted by the lane or lanes of reagent.

81. A method for determining the effect of a plurality of candidate compounds on a monolayer of immobilized cells, comprising:
(a) immobilizing the cells on a target region of a substrate surface;
(b) placing the cells within a flow passage defined at least in part by the substrate surface; and
(c) introducing a plurality of fluids each in contiguous laminar flow through the flow passage, each fluid forming a fluid lane containing a candidate compound downstream from the inlet over a portion of the target region, thereby exposing the cells to the candidate compounds,
wherein at least on fluid is introduced directly into the flow passage in a direction noncoplanar to the substrate surface.

82. The method of claim 81, further comprising, after step (b) and before and during step (c), (b′) maintaining a carrier fluid in contiguous laminar flow through the flow passage and over the target region.
